(19) United States
(12) Patent Application Publication
(10) Pub. No.: US 2008/0269628 A1
(43) Pub. Date: Oct. 30, 2008

(54) Denoising and Artifact Rejection for Cardiac Signal in a Sensis System

(75) Inventors: Detlef W. Koertge, Carpentersville, IL (US); Hongxuan Zhang, Schaumburg, IL (US); Bryon Pelzek, Chicago, IL (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc., Malvern, PA (US)

(21) Appl. No.: 11/831,143

(22) Filed: Jul. 31, 2007

Related U.S. Application Data
(60) Provisional application No. 60/913,905, filed on Apr. 25, 2007.

(51) Int. Cl. A61B 5/0402

(52) U.S. Cl. 600/523

(57) ABSTRACT

A method and apparatus for denoising and rejecting artifacts from cardiac signals, having the steps of accepting a cardiac signal from a patient, subjecting the cardiac signal from the patient to a frequency band width controllable choke to separate the cardiac signal into predefined frequencies, filtering each of the predefined frequencies to remove dynamic common noise, joining each of the predefined frequencies into a cardiac signal without the dynamic common noise, and providing a feedback control to the filtering of each of the predefined frequencies.

### CROSS-REFERENCE TO RELATED APPLICATIONS

This is a United States non-provisional application of U.S. provisional patent application Ser. No. 60/913,905 the entirety of which application is incorporated by reference herein.

### FIELD OF THE INVENTION

The present invention relates to noise and artifact interference reduction. More specifically, the invention provides a method and apparatus to suppress noise and artifacts encountered during evaluation of cardiac signals for medical patients.

### BACKGROUND INFORMATION

Cardiac monitoring equipment can use different approaches and strategies for noise cancellation or reduction and artifact reduction. These conventional approaches include notch filtering for 50/60 Hertz electrical artifacts or low pass filtering for high frequency emission noise. These conventional approaches, however, have several shortcomings that are troublesome for medical personnel performing evaluations.

A first drawback to conventional approaches is that frequency analysis based filtering techniques can not efficiently remove common mode noise and artifact interference that share the same frequency band with cardiac signals (commonly known as overlapping signals).

A second drawback to conventional approaches is that fixed low or high frequency band pass filtering in current denoising and artifact rejection methods can not effectively track and cancel dynamic noise and artifacts (especially broad band noise and semi-white noise), such as voltage/current leakage noise generated from use of bovie knife and cardiac ablators.

Another drawback to conventional approaches is that these methods are designed for linear signal processing and analysis that may not effectively reduce the non-linear and non-stationary noise and artifacts for the cardiac signals.

A further drawback to conventional approaches is that denoising methods do not have enough intrinsic data analysis and characterization of the noise and interference in the cardiac signals which greatly limit the application and efficiency of the noise removal and artifact rejection.

There is a need to provide a method and apparatus of denoising signals and performing artifact rejection related to cardiac signals from medical patients, wherein the signals and artifacts removed overlap with the cardiac signals.

There is a further need to provide a method and apparatus that can effectively track and cancel dynamic noise and artifacts such as voltage/current leakage noise generated from use of medical instruments such as bovie knife and cardiac ablators.

There is also a need to provide a method and apparatus that can evaluate and process non-linear and non-stationary noise as well as artifacts for cardiac signals. There is a further need to provide a method and apparatus that provides sufficient intrinsic data analysis and characterization of the noise and interference in the cardiac signals to overcome the conventional method limitations for efficiency of the noise removal and artifact rejection

### SUMMARY

It is therefore an objective to provide a method and apparatus of denoising signals and performing artifact rejection related to cardiac signals from medical patients, wherein the signals and artifacts removed overlap with the cardiac signals.

It is also an objective to provide a method and apparatus that can effectively track and cancel dynamic noise and artifacts such as voltage/current leakage noise generated from use of medical instruments such as bovie knife and cardiac ablators.

It is a further objective to provide a method and apparatus that can evaluate and process nonlinear and non-stationary noise as well as artifacts for cardiac signals.

It is a still further objective to provide a method and apparatus that provides sufficient intrinsic data analysis and characterization of the noise and interference in the cardiac signals to overcome the conventional methods limitations for efficiency of the noise removal and artifact rejection.

The objectives achieved as illustrated and described. An embodiment of the invention provides a method for denoising and rejecting artifacts from cardiac signals, comprising the steps of accepting a cardiac signal from a patient, separating the cardiac signal into predefined frequencies, filtering each of the predefined frequencies to remove dynamic noise, joining each of the predefined frequencies into a cardiac signal without the dynamic noise, and providing a feedback control to the filtering of each of the predefined frequencies.

The filtering of each of the predefined frequencies to remove dynamic noise is accomplished by identification of interference signals from medical instruments and elimination of noise related to the medical instruments. The method may also be performed such that the dynamic common noise removed is a non-linear signal.

In another embodiment of the invention, the controllable choke separating the signals from the patient is controlled through a computer programmable selection that decreases common mode noise. The filtering of each of the predefined frequencies may be accomplished through empirical mode decomposition processing.

An embodiment of the invention also provides a method for denoising and rejecting artifacts from cardiac signals, comprising the steps of accepting a cardiac signal from a patient, separating the cardiac signal from the patient into predefined frequencies, filtering each of the predefined frequencies to remove dynamic noise, and joining each of the predefined frequencies into a cardiac signal without the dynamic noise. The filtering of each of the predefined frequencies may be accomplished through empirical mode decomposition processing.

In another embodiment, the filtering of each of the predefined frequencies to remove common mode noise is accomplished by identification of interference signals from medical instruments and elimination of noise related to the medical instruments. The dynamic noise removed may be a non-linear signal.

In another embodiment of the invention a program storage device readable by machine, tangibly embodying a program of instructions executable by the machine to perform method steps for denoising and rejecting artifacts from cardiac signals, comprising the steps of accepting a cardiac signal from a patient, separating the cardiac signal from the patient into predefined frequencies; filtering each of the predefined frequencies to remove dynamic noise, joining each of the predefined frequencies into a cardiac signal without the dynamic noise, and providing a feedback control to the filtering of each of the predefined frequencies.

In an embodiment of the invention, the filtering of each of the predefined frequencies is through empirical mode decomposition processing. In another embodiment of the invention, the filtering of each of the predefined frequencies to remove dynamic noise is accomplished by identification of interference signals from medical instruments and elimination of noise related to the medical instruments. Additionally, the dynamic noise removed is a non-linear signal.

In another exemplary embodiment of the invention, a program storage device readable by machine, tangibly embodying a program of instructions executable by the machine to perform method steps for denoising and rejecting artifacts from cardiac signals is presented. The method accomplished comprises accepting a cardiac signal from a patient, separating the cardiac signal from the patient into predefined frequencies, filtering each of the predefined frequencies to remove dynamic common noise, and joining each of the predefined frequencies into a cardiac signal without the dynamic noise. The filtering of each of the predefined frequencies is through empirical mode decomposition processing. The filtering of each of the predefined frequencies to remove dynamic noise is accomplished by identification of interference signals from medical instruments and elimination of noise related to the medical instruments. The dynamic common noise removed is a non-linear signal.

In an additional exemplary embodiment, the controllable choke separating the signals from the patient is controlled through a computer programmable selection that decreases common mode noise.

An embodiment of the present invention also provides an apparatus for denoising signals from a medical patient, comprising a frequency band width controllable choke, the choke configured to accept signals from the medical patient and separate the signal into defined frequencies, at least one filter to accept the defined frequencies produced by the frequency band width controllable choke, and a feedback control connected to the at least one filter. The apparatus may further comprise a software control and calibration arrangement connected to the frequency band width controllable choke, the software control and calibration arrangement configured to control the choke.

### BRIEF DESCRIPTION OF THE DRAWINGS

FIG. 1 is a signal denoising/artifact rejection method and system structure.

FIG. 2 is a hardware based filtering apparatus for a frequency band width controllable choke for dynamic common noise and adaptive tunable frequency band programmer.

FIG. 3 is a frequency band controlling method for adaptive tunable frequency band programmer.

FIG. 4 is a EMD algorithm based signal decomposition and reconstruction.

FIG. 5 is a EMD based signal decomposition and reconstruction of the algorithm of FIG. 4.

### DETAILED DESCRIPTION

An embodiment of the invention provides an efficient method 100 and apparatus 200 for cardiac signal denoising and artifact rejection. The embodiment of the invention provides both adaptive programmable hardware based filters 208, 210 and 212 as well as signal decomposition/reconstruction. In the invention, dynamic noise is defined by an amplitude/frequency/energy distributions of noise that is/are changeable. In an embodiment of the present invention, the adaptive multi-frequency band (at least two frequency band filters) and automatic close-loop (feedback) are used to achieve real-time gain adjustment of the multi-frequency band and to obtain the best signal noise ratio. Hence there is an optimization issue for the multi-frequency band control:

Φ(signal)=f(A₁+A₂+ . . . +Aₙ)

Φ() is the function value of the signal noise ratio; f() is the function to calculate and summarize the signal; Aᵢ is the gain of the i^{th} frequency band. The filtering strategy can achieve the best the signal quality, if Φ(signal) can reach the biggest value. Common mode noise is conducted on all lines in the same direction, such as EMI noise and background/environmental noise.

In patient monitoring, high quality signals are the basis for proper diagnosis and correct medical treatment decision. The minute signals from patient, however, are usually in millivolt (mV) or microvolt (uV) range. These very low level signals are easily distorted and affected by noise, such as electrical emission noise (environmental noise), patient movement and respiration (bio-artifacts), etc.

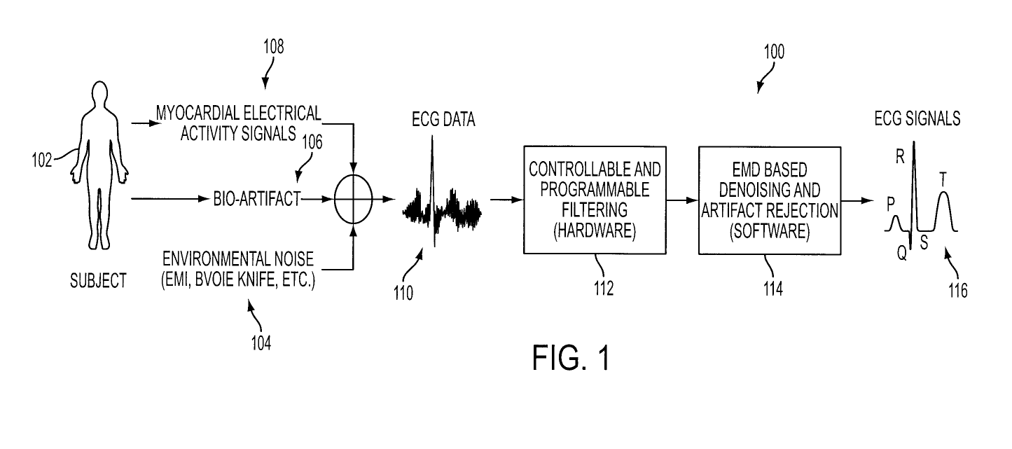

This embodiment of the invention provides both a hardware and software combined method 100 for patient signal denoising, especially for the cardiac electrophysiological activities (ECG signals). Referring to FIG. 1, an embodiment of the invention is provided for conducting denoising and artifact rejection of cardiac signals from a patient. In FIG. 1, a surface ECG signal is used to describe an example of the denoising strategies, but the method 100 presented may comprise applications in any kind of signals, such as pressure signals, intra-cardiac electrograms, invasive and non-invasive. The processing method 100 takes ECG data 110 from a subject 102 for analysis. The ECG data 110 includes myocardial signals 108, bio artifacts 106 and environmental noise 104. The environmental noise 104 may include signals from equipment, such as surgical equipment or general background electrical interference. The total ECG data 110 is then subjected to controllable/programmable filtering 112. In step 114, an EMD based denoising is then conducted on the ECG data 110. The bio artifacts 106 and the environmental noise 104 are removed from the myocardial signals 108 resulting in clean ECG signals 116 that may be analyzed.

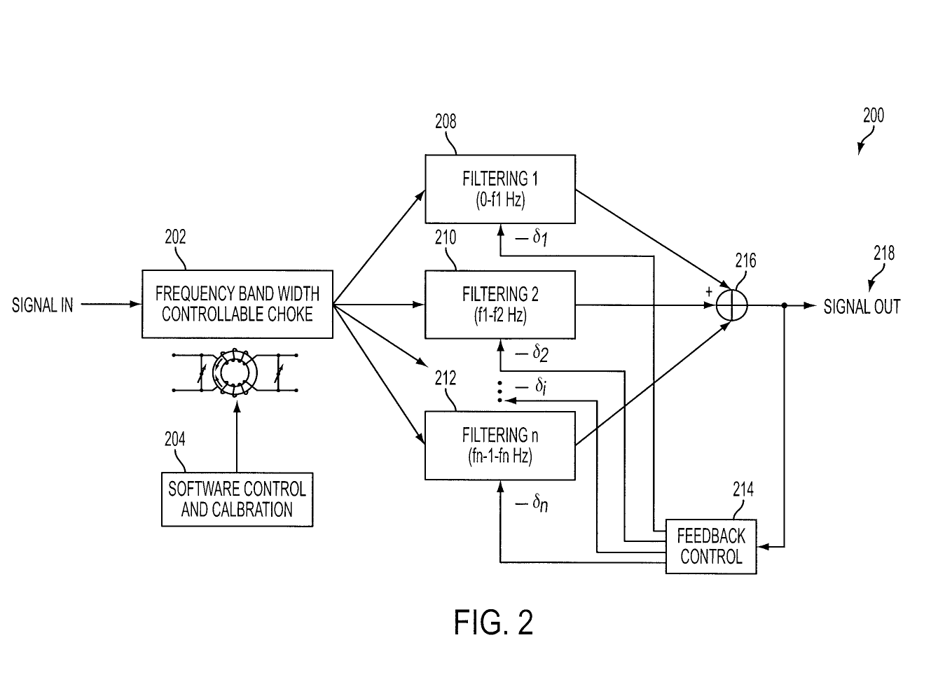

In an embodiment of the invention, a hardware based denoising and artifact rejection apparatus 200 is provided. The apparatus 200 based embodiment includes a common mode noise controller and adaptive tunable frequency and programmer. The common noise controller apparatus 200 receives input in the form of signals, in the present embodiment cardiac signals, and decreases any noise and artifact effects present during a cardiac operation. Referring to FIG. 2, the apparatus 200 includes a frequency based choke 202 and three filters 208, 210, 212 and a feedback control 214.

During cardiac operations, for example, a bovie knife is used to allow the surgeon to accurately modify tissues present within the patient. The usage of the bovie knife, however, generates dynamic noise (electrical signals) to every data acquisition sensor used for patient monitoring. Concurrently, the use of the bovie knife leads to voltage and current leakages to the patient, both of which may shift both signal and GND of the biomedical instrumentation. To complicate matters, the frequency band width of the leaked noise is dynamic and shifting/changing during the operation. This common mode noise, however, is controlled in an embodiment of the invention by a filtering choke 202 that is efficiently controlled and calibrated by software 204. The frequency band width controllable choke 202 technology used in an embodiment of the invention is connected to a feedback controllable apparatus 214 for automatic and adaptive adjustment of the signal frequency band width.

The hardware based filtering apparatus 200 is constructed from two specific parts. The filtering apparatus 200 has a frequency band width controllable choke 202 for dynamic noise previously described in FIG. 1. The apparatus 200 has an adaptive tunable frequency band programmer and controller 204 that can decrease the effects of the common mode noise in some specific frequency band width.

The adaptive tunable frequency programmer in the filtering and denoising hardware arrangement 200 adaptively control signals as well as noise in different bandwidths. In an embodiment of the invention, the ECG signal from the patient and noise generating devices has a frequency band of 0-200 Hz. The frequency band controller greatly decreases the noise in specific bands, such as 50-60 Hz, without attenuating ECG signals in other frequency bands. Based on the feedback of the signal that is provided, the feedback control arrangement 214 analyzes the signal to noise ratio (SNR) of different frequency bands and adjusts the filtering parameters of noisy band. In the illustrated embodiment, there are three bands that are evaluated. After filtering, the signals are combined 216 to produce a signal out 218.

The tunable techniques used in the exemplary embodiment presented are implemented by the hardware as a closed loop for automatic feedback control. Concurrently the filtering parameter and feedback weight δᵢ are programmed and controlled from the firmware on board or software in the PC (application software).

By adjusting the signal and noise level of different frequency bands, the SNR of the output signal 218 is greatly enhanced compared to non-filtered signals 206. The high quality output signal is achieved by sacrificing the signal in the noisy frequency band. The frequency band of the filters, Δfᵢ, in the exemplary embodiment can be tuned and adjusted according to the signal type and application.

The adaptive tunable frequency band programmer based denoising strategy is very useful for removing common mode noise in the specific frequency band, such as the ablator noise (450-500 KHz) and power electrical interference (50-60 Hz). Comparing the results of the invention to notch filtering techniques, the adaptive tunable frequency band filtering is more flexible and stability of the filtering is high.

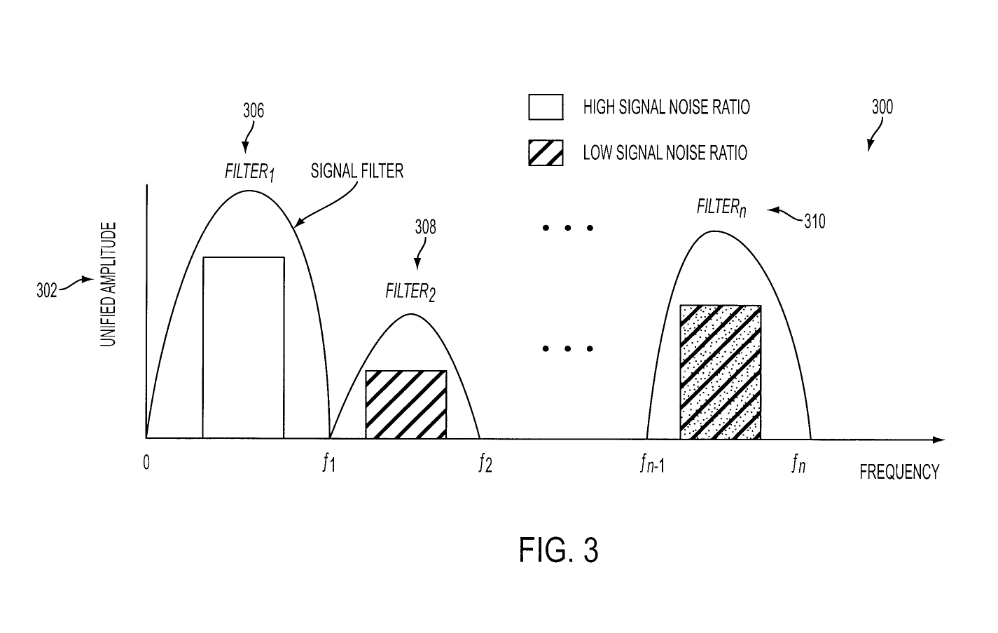

Referring to FIG. 3, a graph 300 of unified amplitude 302 verses frequency 304 of signals for an individual is presented. The frequency band controlling strategies of the adaptive tunable frequency band programmer is illustrated. As provided in FIG. 3, the common mode noise is mainly focusing in the frequency band f₁-f₂ and hence the adaptive feedback controller adjusts the parameter of Filter 2 308 to decrease the noise and artifact effect. By feedback tuning, the output signal quality and SNR are greatly enhanced. As provided with Filter 1 306, a high signal to noise ratio is presented, therefore no adjustments are made for these frequencies. For Filter 3 310, a medium signal to noise ratio is present, therefore no feedback controller adjustment is performed.

The invention also provides a software (signal processing algorithm) based signal filtering method. The signal processing algorithm in the exemplary embodiment of the present invention is an empirical mode function decomposition and reconstruction. Empirical Mode Decomposition (EMD) is a signal processing method for analyzing nonlinear and non-stationary time series. (For example, bovie knife and patient movements always generate non stationary noise and artifacts).

The method of the exemplary embodiment utilizes an EMD algorithm to obtain the decomposed signal components, which may come from the cardiac signals, bio-artifacts, environmental noise, etc. By analyzing the EMD components and sub-signals, the noise based components can be removed prior to EMD signal reconstruction. Hence, the signal to noise ratio of the reconstructed cardiac signal is greatly improved.

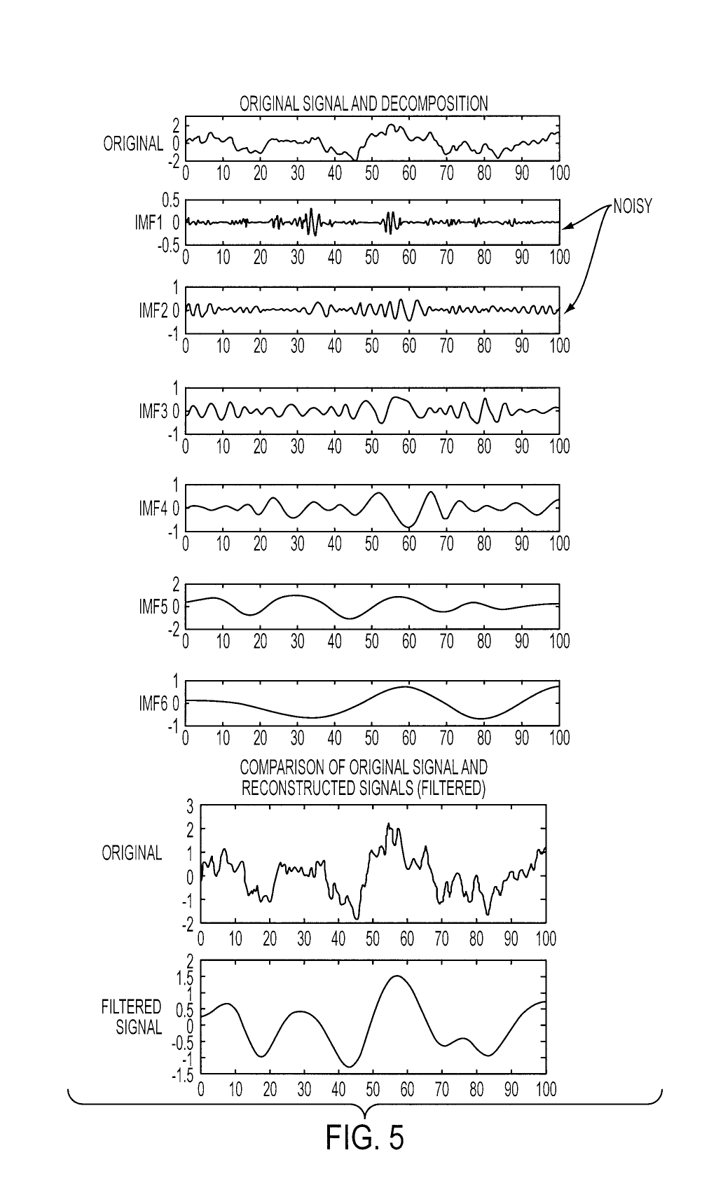

FIG. 5 illustrates an example of the EMD algorithm based signal decomposition and reconstruction. The EMD based signal denoising and artifact rejection are not based on frequency or time analysis, but intrinsic signal oscillators and generators. Although described as providing an EMD algorithm based signal decomposition, other algorithms may be used, including, but not limited to independent component analysis (ICA), primary component analysis (PCA), etc. These exemplary types of signal processing algorithms and theories may be also be used for noise removal.

A first step of data analysis is visual examination of the data. From this examination, different scales are identified by a time lapse between the successive alternations of local maxima and minima; and by time lapse between the successive zero crossings.

The interlaced local extrema and zero crossings produce a complicated data output with one undulation superimposed on another, and they, in turn, are riding on other undulations. Each of these undulations defines a characteristic scale of the data. The exemplary embodiment of the invention adopts a time lapse between successive extrema as the definition of the time scale for the intrinsic oscillatory mode. This is accomplished as it gives a fine resolution of the oscillatory modes and also can be applied to data with a non-zero mean, either all positive or all negative values, without zero crossings. The decomposition procedure is adaptive, data-driven, therefore, highly efficient. A systematic method to extract the intrinsic mode functions (IMFs) or component, designated as the sifting process, is presented to accomplish noise and artifact reduction.

EMD methods according to an embodiment of the invention provide strategies to automatically identify the relevant IMFs that contribute to the slow-varying trend in the data. These methods greatly decrease the time consuming of the signal analysis and enhance EMD method application efficiency, especially in the cardiac signal denoising and artifact rejection. Additionally, signal pre-processing, such as filtering, of the decomposed signal components before the reconstruction may be needed and helpful for better SNR and signal quality.

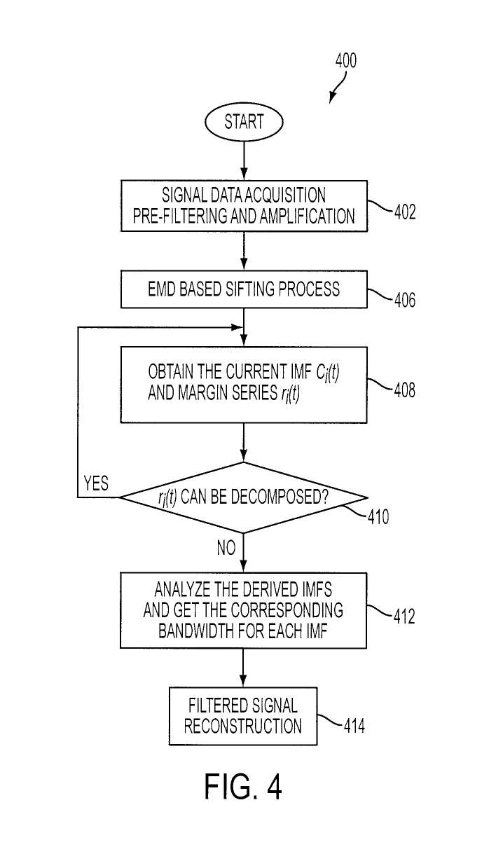

Referring to FIG. 4, the procedure 400 of EMD decomposition is provided, according to an embodiment of the invention, that specifies if the number of maxima or minima of data series X(t) is larger than the number of up-zero (or down-zero) crossing points by two, then the series needs to be forced to be stationary. The detailed procedures are as follows:

The method is started 402 from acquiring signal data from a patient 102. Then a EMD based sifting process is accomplished 406. The sifting process is accomplished by obtaining a current IMF 408 (noise components) of the signal. To achieve this, the current IMF noise components, the data must be evaluated such that:

(i) Pick out all of the maxima of the series X(t) and calculate the upper envelop with cubic spline function.
(ii) Pick out all of the minima of the series X(t) and calculate the lower envelop with cubic spline function.

Next, in the non-limiting exemplary embodiment of the invention, the mean envelop m₁(t) of the series X(t) is the mean value of the upper and lower envelops. A new series h₁ with low frequency removed is calculated by subtracting the mean envelop from the series X(t):

X(t)−m₁(t)=h₁(t)

In the exemplary embodiment, h₁ is a non-stationary series, so the above procedure must be repeated k times until the mean envelop is approximate to zero, so the first IMF component C₁(t) is obtained:

hₖ₋₁(t)−m₁ₖ(t)=h₁ₖ(t)

C₁(t)=h₁ₖ(t)

The first IMF component represents the highest frequency component of the original series. The second IMF component C₂ (t) is obtained from r₁(t) which is calculated by subtracting the first IMF component from series X(t). Such procedure is repeated until the last margin series rₙ(t) cannot be decomposed further 410, here rₙ(t) represents the mean value or trend of the original series.

r₁(t)−C₂(t)=r₂(t), . . . , rₙ(t)−Cₙ(t)=rₙ(t)

Finally, the original series is presented by a sum of the IMF components and a mean value or trend, as provided in step 412:

$$X ( t ) = {\sum }_{j = 1}^{n} {C}_{j} ( t ) + {r}_{n} ( t )$$

Since every IMF component (IMFi) is a series with a definite characteristic scale, the sifting procedure actually decomposes the original series to a superimposition of waves with various scales. Every IMF component can be either linear or nonlinear. Lastly, the filtered signal is reconstructed 414.

The embodiment of the invention provides a method and apparatus that allows for superior patient protection by decreasing power leakage and electromagnetic interference that patients are subjected to.

The embodiment of the current invention provides several advantages over conventional techniques, including providing a controllable choke 202 based common noise rejection to reduce dynamic EMI noise.

The embodiment of the invention also provides an adaptive filtering technique that allows the user to enter a frequency band for analysis to decrease color noise from the signal of interest. Furthermore, the embodiment of the present invention provides a cardiac electrophysiological activity extraction via intrinsic signal (resources) decomposition and reconstruction, described as Empirical Mode Decomposition (EMD) processing, to cancel the bio-artifacts and noise.

In the foregoing specification, the invention has been described with reference to specific exemplary embodiments thereof. It will, however, be evident that various modifications and changes may be made thereunto without departing from the broader spirit and scope of the invention as set forth in the appended claims. The specification and drawings are accordingly to be regarded in an illustrative rather than in a restrictive sense.

What is claimed is:

1. A method for denoising and rejecting artifacts from cardiac signals, comprising:
accepting a cardiac signal from a patient;
separating the cardiac signal into predefined frequencies;
filtering each of the predefined frequencies to remove dynamic noise;
joining each of the predefined frequencies into a cardiac signal without the dynamic noise; and
providing a feedback control to the filtering of each of the predefined frequencies.

2. The method according to claim 1, wherein the filtering of each of the predefined frequencies to remove dynamic noise is accomplished by identification of interference signals from medical instruments and elimination of noise related to the medical instruments.

3. The method according to claim 1, wherein the dynamic noise removed is a non-linear signal.

4. The method according to claim 1, wherein the signals from the patient are controlled through a computer programmable selection that decreases common mode noise.

5. The method according to claim 1, wherein the filtering of each of the predefined frequencies is through empirical mode decomposition processing.

6. A method for denoising and rejecting artifacts from cardiac signals, comprising:
accepting a cardiac signal from a patient;
separating the cardiac signal from the patient into predefined frequencies;
filtering each of the predefined frequencies to remove dynamic noise; and
joining each of the predefined frequencies into a cardiac signal without the dynamic noise.

7. The method according to claim 6, wherein the filtering of each of the predefined frequencies is through empirical mode decomposition processing.

8. The method according to claim 6, wherein the filtering of each of the predefined frequencies to remove dynamic noise is accomplished by identification of interference signals from medical instruments and elimination of noise related to the medical instruments.

9. The method according to claim 6, wherein the dynamic noise removed is a non-linear signal.

10. A program storage device readable by machine, tangibly embodying a program of instructions executable by the machine to perform method steps for denoising and rejecting artifacts from cardiac signals, comprising:
accepting a cardiac signal from a patient;
separating the cardiac signal from the patient into predefined frequencies;
filtering each of the predefined frequencies to remove dynamic noise;
joining each of the predefined frequencies into a cardiac signal without the dynamic noise; and
providing a feedback control to the filtering of each of the predefined frequencies.

11. The device according to claim 10, wherein the filtering of each of the predefined frequencies is through empirical mode decomposition processing.

12. The device according to claim 10, wherein the filtering of each of the predefined frequencies to remove dynamic noise is accomplished by identification of interference signals from medical instruments and elimination of noise related to the medical instruments.

13. The device according to claim 10, wherein the dynamic noise removed is a non-linear signal.

14. A program storage device readable by machine, tangibly embodying a program of instructions executable by the machine to perform method steps for denoising and rejecting artifacts from cardiac signals, comprising:
accepting a cardiac signal from a patient;
separating the cardiac signal from the patient into predefined frequencies;
filtering each of the predefined frequencies to remove dynamic noise; and
joining each of the predefined frequencies into a cardiac signal without the dynamic noise.

15. The method according to claim 14, wherein the filtering of each of the predefined frequencies is through empirical mode decomposition processing.

16. The method according to claim 15, wherein the filtering of each of the predefined frequencies to remove dynamic noise is accomplished by identification of interference signals from medical instruments and elimination of noise related to the medical instruments.

17. The method according to claim 15, wherein the dynamic noise removed is a non-linear signal.

18. An apparatus for denoising signals from a medical patient, comprising:
a frequency band width controllable choke, the choke configured to accept signals from the medical patient and separate the signal into defined frequencies;
at least one filter to accept the defined frequencies produced by the frequency band width controllable choke; and
a feedback control connected to the at least one filter.

19. The apparatus according to claim 18, further comprising:
a software control and calibration arrangement connected to the frequency band width controllable choke, the software control and calibration arrangement configured to control the choke.
